(19) United States
(12) Patent Application Publication
(10) Pub. No.: US 2013/0197387 A1
(43) Pub. Date: Aug. 1, 2013

(54) System and Method for Measuring Movement of a Body Part

(71) Applicant: Rest Devices, Inc., Boston, MA (US)

(72) Inventors: Thomas Lipoma, Watsonville, CA (US); Pablo Bello, Cambridge, MA (US); Carson Darling, Bellingham, WA (US); Chao He, Newton, MA (US)

(73) Assignee: REST DEVICES, INC., Boston, MA (US)

(21) Appl. No.: 13/745,098

(22) Filed: Jan. 18, 2013

Related U.S. Application Data
(60) Provisional application No. 61/587,838, filed on Jan. 18, 2012.
(60) Provisional application No. 61/713,177, filed on Oct. 12, 2012.

(51) Int. Cl. A61B 5/00; A61B 5/08

(52) U.S. Cl. CPC A61B 5/6804; A61B 5/0004; A61B 5/746; A61B 5/7475; A61B 5/0826
USPC 600/535; 600/595; 427/2.12

(57) ABSTRACT

A device and method for measuring movement of a body part of a subject. The method illustratively includes mounting on the body part, via at least one dielectric, at least two conductive elements in a manner as to define a fixed spacing between the elements and wherein the distance between the conductive elements and the body part varies with movement of the body part. Changes are measured in capacitance between the elements and the body part that result from movement of the body part. Data associated with the changes in capacitance is transmitted to a personal computing device

### CROSS-REFERENCE TO RELATED APPLICATIONS

This application claims priority from U.S. Provisional Patent Application No. 61/587,838, filed Jan. 18, 2012 and U.S. Provisional Patent Application No. 61/713,177 filed Oct. 12, 2012. Each of the above-described applications is hereby incorporated herein by reference in its entirety.

### TECHNICAL FIELD

The present invention relates to monitoring systems, devices and methodologies, and more particularly, to systems and methods for measuring movement or other parameters associated with a body part, such as for respiratory monitoring.

### BACKGROUND ART

In many settings, it is difficult to accurately measure a person's respiratory effort. In the sleep field, it is very common to use an inductance measuring band placed around the thorax to detect a change in chest diameter. Further devices employ motion tracking cameras to detect movement of a tracker on the chest or of the chest surface itself Many of these devices are either uncomfortable or difficult to setup and calibrate. In addition, many of these devices are prone to error. Inductance based respiratory bands often slip down a patents chest, requiring a recalibration.

To solve this problem, inductance based respiratory bands are constrained tightly to a person's chest making them very uncomfortable. Other image based sensors such as motion tracking cameras are extremely calibration dependent. For accurate results, motion tracking devices are required to have a very precise setup, usually above a motionless person in the supine position. Because of the setup requirements, these types of devices do not perform well over long periods of time, in unusual situations, or for remote monitoring.

Furthermore, it is known in the prior art to be difficult to repeatedly quantify the level of apnea in a patient. Current state of the art of apnea diagnosis requires a person to sleep for a single night in a sleep lab. Because of the cost of such a study, measurements of apnea typically last a single night. This lowers the effectiveness of a diagnosis as night-to-night variations can't be observed. In addition, a typical apnea diagnosis in a sleep lab requires multiple sensing devices making sleep uncomfortable. Such a study disrupts a person's sleep, limiting the duration of a test.

To solve this problem, many have attempted to create smaller, at home, apnea diagnostic tools. Many incorporate flow measuring sensors attached to the head. While cheaper, these tools are still limited and typically incorporate multiple sensors. In addition, many are severely limited in accuracy.

### SUMMARY OF THE EMBODIMENTS

In accordance with a first embodiment of the invention, there is provided a method for measuring movement of a body part of a subject. In this embodiment the method includes: mounting on the body part, via at least one dielectric, at least two conductive elements in a manner as to define a fixed spacing between the elements and wherein the distance between the conductive elements and the body part varies with movement of the body part; and measuring changes in capacitance between the elements and the body part that result from movement of the body part.

In a related embodiment, measuring changes in capacitance includes measuring changes in a series capacitance along a path that includes each conductive element and the body part. Optionally, the dielectric is an elastic substrate that is mounted on the body part. Also optionally, the elastic substrate is an article of clothing worn by the subject. Alternatively, or in addition, the conductive elements are also mounted to a restraining member that maintains the fixed spacing. In a further related embodiment, the body part is the chest, and movement of the chest is used to measure respiration.

In accordance with another embodiment of the invention, there is provided a device for measuring movement of a body part of a subject. In this embodiment, the device includes: at least one dielectric that is mountable on the body part; at least two conductive elements, mounted on the at least one dielectric in a manner as to define a fixed spacing between the elements; and a capacitive output coupled to the at least two conductive elements, wherein the capacitive output is reflective of capacitance between the elements and the body part; wherein the dielectric is configured so that distance between the conductive elements and the body part varies with movement of the body part so as to produce corresponding changes in the capacitive output.

In a related embodiment, the dielectric is an elastic substrate that is mounted on the body part. Optionally, the elastic substrate is an article of clothing worn by the subject. Alternatively, or in addition, the conductive elements are also mounted to a restraining member that maintains the fixed spacing. In a further related embodiment, the body part is the chest, further comprising a controller, coupled to the capacitive output, and configured to measure respiration as determined from movement of the chest.

In accordance with another embodiment of the invention, a method for monitoring a body part of a subject is presented. The method includes mounting on the body part, via at least one dielectric, at least two conductive elements in a manner as to define a fixed spacing between the elements and wherein the distance between the conductive elements and the body part varies with movement of the body part. Changes in capacitance between the elements and the body part that result from movement of the body part are measured. Data associated with the changes in capacitance are transmitted to a personal computing device.

In accordance with related embodiments of the invention, measuring changes in capacitance may include measuring changes in a series capacitance along a path that includes each conductive element and the body part. The dielectric may be an elastic substrate that is mounted on the body part. The elastic substrate may be an article of clothing worn by the subject. The article of clothing may include infant clothing, such as a bodysuit. The conductive elements may be mounted to a restraining member that maintains the fixed spacing. The body part may be the chest, and movement of the chest may be used to measure respiration.

In accordance with further related embodiments of the invention, transmitting at least one of data and alerts associated with the changes to a personal computing device may include transmitting data associated with the changes to a base station, which may then transfer the data to a network server. The network server may then transmit data associated with the changes to the personal computing device. The data may be analyzed at the network server. Transmitting data associated with the changes to the base station may be performed wirelessly, for example, via a Bluetooth transmitter.

In still further embodiments of the invention, the method may include using one or more sensors to acquire additional data associated with the subject, and transmitting the additional data to the personal computing device. The one or more sensors may include at least one of an accelerometer and/or a temperature sensor.

In yet further embodiments of the invention, the personal computing device may be, without limitation, a phone, a computer, a netbook, a laptop, and/or a tablet. Transmitting data associated with the changes in capacitance to a personal computing device may include transmitting an alert.

In accordance with another embodiment of the invention, a system for monitoring a body part of a subject includes at least one dielectric that is mountable on the body part. At least two conductive elements are mounted on the at least one dielectric in a manner as to define a fixed spacing between the elements. A capacitive output is coupled to the at least two conductive elements, wherein the capacitive output is reflective of capacitance between the elements and the body part. A controller is coupled to the substrate for transmitting data associated with the capacitive output. The dielectric is configured so that distance between the conductive elements and the body part varies with movement of the body part so as to produce corresponding changes in the capacitive output.

In accordance with related embodiments of the invention, the dielectric may be an elastic substrate that is mounted on the body part. The elastic substrate may be an article of clothing worn by the subject. The article of clothing may be infant clothing. The conductive elements may be mounted to a restraining member that maintains the fixed spacing.

In accordance with further related embodiments of the invention, the system may further include a base station and a network server, wherein the controller transmits data associated with the capacitive output to the base station, and wherein the base station transmits the data to the network server. The network server may analyze the data and transmit data associated with the capacitive output to a personal computing device. The network server may be configured to provide an alarm to the personal computing device based, in part, from the data received from the base station. The body part may be the chest, and the network server may be configured to provide to the personal computing device respiration information as determined from movement of the chest. The personal computing device may be a phone, a computer, a netbook, a laptop, and/or a tablet.

In accordance with still further related embodiments of the invention, the system may include one or more sensors to acquire additional data associated with the subject, wherein the controller is configured to wirelessly transmit the additional data. The one or more sensors may include an accelerometer and/or a temperature sensor. The controller may wirelessly transmit data associated with the capacitive output.

In accordance with various embodiments of the invention, a system and methodology for monitoring a body part of a subject includes at least one sensor coupled to an article of clothing, such as infant clothing. A controller is operatively coupled to the at least one sensor, the controller for transmitting data associated with output from the sensor. The controller may transmit data associated with the output from the sensor to a base station, for example, by Bluetooth. The base station may then transmit data associated with output from the sensor to a network server, which may then forward data associated with output from the sensor to a personal computing device. In various embodiments, the controller may wirelessly transmit data (which may include, without limitation, alerts) associated with the output from the sensor directly to the personal computing device.

In accordance with related embodiments of the invention, the network server may analyze the data received from the base station and transmit data associated with the sensor output to a personal computing device. For example, the network server may be configured to provide an alarm to the personal computing device based, in part, from the data received from the base station. The body part may be the chest, and the network server may be configured to provide to the personal computing device respiration information as determined from movement of the chest. The personal computing device may be a phone, a computer, a netbook, a laptop, and/or a tablet. The controller may wirelessly transmit data associated with the sensor output.

In accordance with another embodiment of the invention, a method of monitoring a body part of a subject is provided. The method includes mounting on the body part, via at least one dielectric, at least two conductive elements in a manner wherein the distance between the conductive elements and the body part varies with movement of the body part. Each conductive element includes at least two layers of conductive material bonded together so as to create an interface between the at least two layers that impedes crack propagation from one layer to an adjacent layer. Changes in capacitance between the elements and the body part are measured that result from movement of the body part.

In accordance with related embodiments of the invention, the at least two layers of conductive material may include a first and second film layer, the second film layer deposited onto the first film layer after curing and/or flash curing the first film layer, whereupon the second film layer is cured and/or flash cured so as to form the bond therebetween. One or more of the at least two layers of conductive material may include conductive ink. One of the at least two layers of conductive material may include a material that varies from the other of the at least two layers of conductive material. Measuring changes in capacitance may include measuring changes in a series capacitance along a path that includes each conductive element and the body part. The dielectric may be an elastic substrate that is mounted on the body part. The dielectric may be an article of clothing worn by the subject, such as infant clothing. The body part may be the chest, and movement of the chest is used to measure respiration. The conductive elements may be mounted to a restraining member that maintains a fixed spacing between the conductive elements.

In accordance with further related embodiments of the invention, the method may include transmitting data associated with the measured changes to a base station; transmitting data associated with the measured changes from the base station to a network server; and transmitting data associated with the measured changes to a personal computing device. The personal computing device may be a phone, a computer, a netbook, a laptop, and/or a tablet. The data may be analyzed at the network server (e.g., without any or minimal analysis performed by the controller and/or base station). Transmitting data associated with the changes in capacitance to a personal computing device may include transmitting an alert.

In accordance with still further embodiments of the invention, further measurements associated with the subject may be acquired using one or more additional sensors, such as an accelerometer, a moisture sensor and/or a temperature sensor.

In accordance with another embodiment of the invention, a system for monitoring a body part of a subject includes at least one dielectric that is mountable on the body part. At least two conductive elements are coupled to the at least one dielectric, the at least two conductive elements including a capacitive output reflective of capacitance between the elements and the body part. Each conductive element includes at least two layers of conductive material bonded together so as to create an interface between the at least two layers that impedes crack propagation from one layer to an adjacent layer. The dielectric is configured so that distance between the conductive elements and the body part varies with movement of the body part so as to produce corresponding changes in the capacitive output.

In accordance with related embodiments of the invention, the at least two layers of conductive material may include a first and second film layer, the second film layer deposited onto the first film layer after curing and/or flash curing the first film layer. The second film layer is cured and/or flash cured so as to form the bond therebetween. One or more of the at least two layers of conductive material may include conductive ink. One of the at least two layers of conductive material may include a material that varies from the other of the at least two layers of conductive material. The dielectric may be an elastic substrate that is mounted on the body part. The elastic substrate may be an article of clothing worn by the subject, such as infant clothing. The conductive elements may also be mounted to a restraining member that maintains a fixed spacing between the conductive elements.

In accordance with further related embodiments of the invention, a controller may be coupled to the substrate for transmitting data associated with the capacitive output. The system may further include a base station and a network server, wherein the controller transmits data associated with the capacitive output to the base station, and the base station transmits the data to the network server, the network server for analyzing the data and transmitting data associated with the capacitive output to a personal computing device. The network server may be configured to provide an alarm to the personal computing device based off the data received from the base station.

In accordance with still further related embodiments of the invention, the body part may be the chest, with the network server configured to provide to the personal computing device respiration information as determined from movement of the chest. The system may include the personal computing device, which may be a phone, a computer, a netbook, a laptop, and/or a tablet.

In yet further embodiments of the invention, the system may include one or more additional sensors to acquire additional data associated with the subject, wherein the controller is configured to wirelessly transmit the additional data. The additional sensors may include an accelerometer, a moisture sensor and/or a temperature sensor.

In accordance with another embodiment of the invention, a method and system for monitoring a body part of a subject includes an article of infant clothing. At least one sensor is coupled to the article of clothing, the sensor providing output resulting from movement of the body part, the movement associated with respiration. A controller is coupled to the substrate and operatively coupled to the at least one sensor, the controller for transmitting data associated with output from the sensor. A base station transmits data received from the controller associated with output from the sensor. A network server receives and analyzes data from the base station associated with output from the sensor and determines a condition, such as apnea and SIDS. The network server transmits alerts pertaining to the condition. A personal computing device having a user interface receiving alerts from the network server pertaining to the condition, and providing the alerts to a user via the user interface.

In accordance with related embodiments of the invention, each conductive element may include at least two layers of conductive material bonded together so as to create an interface between the at least two layers that impedes crack propagation from one layer to an adjacent layer. The dielectric may be configured so that distance between the conductive elements and the body part varies with movement of the body part so as to produce corresponding changes in the capacitive output.

In accordance with further related embodiments of the invention, the at least two layers of conductive material may include a first and second film layer, the second film layer deposited onto the first film layer after curing and/or flash curing the first film layer. The second film layer is cured and/or flash cured so as to form the bond therebetween. One or more of the at least two layers of conductive material may include conductive ink. One of the at least two layers of conductive material may include a material that varies from the other of the at least two layers of conductive material. The dielectric may be an elastic substrate that is mounted on the body part. The elastic substrate may be an article of clothing worn by the subject, such as infant clothing. The conductive elements may also be mounted to a restraining member that maintains a fixed spacing between the conductive elements.

In accordance with another embodiment of the invention, a method of fabricating a sensor includes coupling to a dielectric at least two conductive elements forming a capacitive output. Each conductive element is formed by depositing a first layer of conductive material, the first layer including solvent. The first layer is at least partially cured such that at least a portion of the solvent in the first layer is removed. A second layer of conductive material is deposited on the at least partially cured first layer, the second layer including solvent. The second layer is at least partially cured such that at least a portion of the solvent in the second layer is removed, wherein an interface is formed between the first and second layer that impedes crack propagation from the first layer to the second layer.

In accordance with related embodiments of the invention, a controller may be coupled to the capacitive output, the controller for receiving the capacitive output and transmitting data associated with the capacitive output. The controller may be fixedly or removably coupled to the dielectric. The conductive material may be a conductive ink. The dielectric may be an article of clothing, such as infant clothing, such as a bodysuit. The bodysuit may cover the chest of an infant having a chest length, and wherein the length of the first layer of conductive material is more than 50% the width of the chest. The dielectric may be elastic such that respiration of the infant causes the dielectric to stretch. The dielectric may be a diaper or an adhesive bandage. The method may further include depositing the first layer on a substrate, and coupling the substrate to the dielectric. The substrate may be a heat transfer t-shirt vinyl. The first layer may be deposited directly on the dielectric.

In accordance with another embodiment of the invention, a method for determining the occurrence of an abnormal respiratory event in a subject is provided. The method includes receiving at least one time-varying electrical input signal characterizing respiratory activity. A time-varying segmented electrical following signal is generated wherein each segment thereof corresponds to a segment of the input signal. The following signal is electronically monitored to identify a specific segment thereof having a duration in excess of a threshold, and if the specific segment has been identified, then an abnormal respiratory event is stored associated with a segment in the input signal corresponding to the specific segment.

In accordance with related embodiments of the invention, generating the following signal may include determining a peak in the input signal, and upon determining a peak in the input signal, generating a segment of the following signal. The method may include determining if the input signal rises above the corresponding following signal within the threshold duration, wherein no abnormal respiratory event is associated with the segment. Upon determining that the input signal rises above the corresponding following signal within the threshold duration, the method further may further include ending the segment, determining a peak in the input signal, and upon determining a peak in the input signal, generating another segment of the following signal.

In accordance with further related embodiments of the invention, the segment of the following signal may have an amplitude that decreases over at least a portion of time. This decrease in amplitude may be based, at least in part, on the input signal. The amplitude of the following signal may be composed of, at least in part, the envelope of the input signal. The amplitude of the following signal may decrease linearly over at least a portion of time, and/or may decrease exponentially over at least a portion of time. The amplitude of the following signal may initially decrease and then stop decreasing after an amount of time. The amplitude may not decrease below a predetermined value. The threshold may be a predetermined value or determined from the input signal. An alert may be generated at a user interface upon storing the abnormal respiratory event.

In accordance with another embodiment of the invention, a computer program product for determining the occurrence of an abnormal respiratory event in a subject is provided. The computer program product including a non-transitory computer usable medium having computer readable program code thereon. The computer readable program code including program code for receiving at least one time-varying input signal characterizing respiratory activity; program code for generating a time-varying segmented electrical following signal wherein each segment thereof corresponds to a segment of the input signal; and program code for monitoring the following signal to identify a specific segment thereof having a duration in excess of a threshold, and if the specific segment has been identified, then storing an abnormal respiratory event associated with a segment in the input signal corresponding to the specific segment.

In accordance with related embodiments of the invention, the program code for generating the following signal may include program code for determining a peak in the input signal, and program code for, upon determining a peak in the input signal, generating a segment of the following signal. The computer program product may further include program code for determining if the input signal rises above the corresponding following signal within the threshold duration, wherein no abnormal respiratory event is associated with the segment. The computer program product may include program code for, upon determining that the input signal rises above the corresponding following signal within the threshold duration, ending the segment, determining a peak in the input signal, and upon determining a peak in the input signal, generating another segment of the following signal. A segment of the following signal may have an amplitude that decreases over at least a portion of time. The decrease may be linear or exponential. The decrease in the amplitude may be based at least in part, on the input signal. The amplitude may initially decrease and then stop decreasing after an amount of time. The amplitude may not decrease below a predetermined value. The amplitude may include, at least in part, the envelope of the input signal. The threshold may be determined from the input signal. The product may include program code for generating an alert at a user interface upon storing the abnormal respiratory event.

In accordance with another embodiment of the invention, a system for determining the occurrence of an abnormal respiratory event in a subject is provided. The system includes a controller having an input for receiving at least one time-varying input signal characterizing respiratory activity. The controller is configured to generate a time-varying segmented electrical following signal wherein each segment thereof corresponds to a segment of the input signal. Furthermore, the controller is configured to monitor the following signal to identify a specific segment thereof having a duration in excess of a threshold. If the specific segment has been identified, the controller stores an abnormal respiratory event associated with a segment in the input signal corresponding to the specific segment.

In accordance with a related embodiment of the invention, the system may include at least one dielectric that is mountable on a body part of the subject. At least two conductive elements are coupled to the at least one dielectric. The at least two conductive elements include a capacitive output reflective of capacitance between the elements and the body part and characterizing respiratory activity. The dielectric is configured so that distance between the conductive elements and the body part varies with movement of the body part so as to produce corresponding changes in the capacitive output, wherein the capacitive output is the time-varying input signal received at the input of the controller.

In accordance with further related embodiments of the invention, the dielectric may be an article of clothing, an adhesive bandage or a diaper. The controller may be configured to determine a peak in the input signal, and generate a segment of the following signal. The controller may be configured to determine if the input signal rises above the corresponding following signal within the threshold duration, wherein no abnormal respiratory event is associated with the segment. Upon determining that the input signal rises above the corresponding following signal within the threshold duration, the controller may be further configured to end the segment, determine a peak in the input signal, and upon determining a peak in the input signal, generate another segment of the following signal. The amplitude of a segment of the following signal may have an amplitude, the amplitude decreasing over at least a portion of time. The amplitude may decrease over at least a portion of time, based at least in part, on the input signal. The amplitude may include, at least in part, the envelope of the input signal. The amplitude may decrease linearly and/or exponentially over at least a portion of time. The amplitude may initially decrease, and stop decreasing after an amount of time. The amplitude may not decrease below a predetermined value. The threshold may be a predetermined signal and/or be based, at least in part, on the input signal.

In accordance with another embodiment of the invention, a method for monitoring a body part of a subject is provided. The method includes mounting an adhesive bandage on the body part, the adhesive bandage including at least two conductive elements. Changes in capacitance between the elements and the body part are measured that result from movement of the body part.

In accordance with a related embodiment of the invention, the adhesive bandage may include a base layer having a first surface and a second surface opposite the first surface. The at least two conductive elements are coupled to the first surface of the base layer. First and second adhesive are attached to the second surface at a first end and a second end of the adhesive bandage, respectively, the adhesives for attaching to the body part. The adhesive bandage further including a compressible material positioned between the first and second adhesives.

In accordance with related embodiments of the invention, measuring changes in capacitance may include measuring changes in a series capacitance along a path that includes each conductive element and the body part. Movement of the body part may stretch the baselayer causing the compressible material to compress so as to bring the conductive elements closer to the body thereby increasing the capacitance. The body part may be the chest, and movement of the chest is used to measure respiration. The method may further include transmitting the measured changes in capacitance.

In accordance with another embodiment of the invention, a system for monitoring a body part of a subject includes an adhesive bandage that is mountable on the body part. The adhesive bandage includes at least two conductive elements. A capacitive output is coupled to the at least two conductive elements, wherein the capacitive output is reflective of capacitance between the elements and the body part. The distance between the conductive elements and the body part varies with movement of the body part so as to produce corresponding changes in the capacitive output.

In accordance with related embodiment of the invention, the adhesive bandage may include a base layer having a first surface and a second surface opposite the first surface. The at least two conductive elements are coupled to the first surface of the base layer. A first and second adhesive is attached to the second surface at a first end and a second end of the band aid, respectively, the adhesives for attaching to the body part. The adhesive bandage further includes a compressible material positioned between the first and second adhesives.

In accordance with further related embodiments of the invention, the baselayer is elastic, and movement of the body part stretches the baselayer causing the compressible material to compress so as to bring the conductive elements closer to the body. The system may further include a controller coupled to the adhesive bandage for transmitting data associated with the capacitive output.

In accordance with another embodiment of the invention, a method for detecting wetness associated with a body part of a subject is provided. The method includes mounting on the body part, via at least one dielectric, at least two conductive elements at a location with substantially no body movement relative to the conductive elements. Changes in capacitance are measured between the elements and the body part that result from a wet condition.

In accordance with related embodiments of the invention, measuring changes in capacitance includes measuring changes in a series capacitance along a path that includes each conductive element and the body part. The conductive elements may be mounted to a restraining member that maintains a fixed spacing. The dielectric may be an article of clothing. The article of clothing may be a diaper, and the location of the conductive elements is the groin area. Determining that the diaper is wet may be based, at least in part, on the capacitive output. An alarm may be provided based off of an indication of a wet diaper.

In accordance with another embodiment of the invention, a system for detecting wetness associated with an article of clothing worn by a subject is provided. The method includes mounting at least two conductive elements on the article of clothing at a location associated with substantially no body movement relative to the conductive elements when the article of clothing is worn by the subject. A capacitive output is coupled to the at least two conductive elements, wherein the capacitive output is reflective of capacitance between the elements and the body part, and wherein wetness produces corresponding changes in the capacitive output.

In accordance with related embodiments of the invention, the system further includes a controller, coupled to the capacitive output, and configured to determine whether the article of clothing is wet based, at least in part, on the capacitive output. The controller may be configured to provide an alarm based off of an indication of wetness. The article of clothing may be a diaper, wherein the location of the conductive elements may be the groin area.

### BRIEF DESCRIPTION OF THE DRAWINGS

The foregoing features of embodiments will be more readily understood by reference to the following detailed description, taken with reference to the accompanying drawings, in which:

FIG. 1 is a schematic diagram of an article of clothing including sensors for gathering respiration data according to embodiments of the invention;

FIG. 2 is a top view of a sensor as shown in FIG. 1;

FIG. 3 is a side view of a sensor as show in FIG. 1;

FIG. 4 is a graph showing a sample of respiration data gathered from an embodiment of the sensor shown in FIG. 2 and FIG. 3;

FIG. 5 is a schematic diagram showing a controller operatively coupled to a personal computing device via a base station, internet, and server, in accordance with an embodiment of the invention;

FIG. 6 shows a controller coupled to a bodysuit, in accordance with an embodiment of the invention;

FIG. 7 shows a cross-section of various components associated with a system for monitoring a body part, in accordance with an embodiment of the invention;

FIGS. 8(a-d) show exemplary screen shots that may be displayed on a personal computer device, in accordance with various embodiments of the invention. More particularly, FIG. 8(a) shows a home display mode. FIG. 8(b) shows a live monitor mode. FIG. 8(c) shows an alert settings mode. FIG. 8(d) shows a controller set up mode;

FIG. 9 shows an input signal characteristic of respiration, in accordance with an embodiment of the invention;

FIG. 10 shows an input signal characteristic of respiration that includes an apnea event, in accordance with an embodiment of the invention;

FIG. 11 shows an input signal characteristic of respiration that includes an apnea event, in accordance with an embodiment of the invention;

FIG. 12 shows a flow diagram depicting an exemplary process of detecting an abnormal respiration event, in accordance with an embodiment of the invention;

FIGS. 13(a-c) show exemplary flow diagrams depicting how data is transferred into and out of the server, in accordance with various embodiments of the invention. More particularly, FIG. 13(a) is a flow diagram depicting how data flows from the devices into the server. FIG. 13(b) is a flow diagram showing how the server handles running algorithms, storing data, and storing algorithm outputs. FIG. 13(c) is a flow diagram showing how the server handles communication with personal computing devices and sending alerts to users communicating with the server;

FIG. 14 shows an adhesive bandage that may include one or more sensors, in accordance with an embodiment of the invention;

FIG. 15 shows an exemplary respiration signal that includes a spike caused by sensor degradation, in accordance with an embodiment of the invention.

### DETAILED DESCRIPTION OF SPECIFIC EMBODIMENTS

In illustrative embodiments of the invention, a reliable system and method of monitoring a body part of a subject, such as movement of a chest, is provided. Such embodiments may advantageously be used to provide detection of respiratory events, such as apnea or sudden infant death syndrome (SIDS). Further embodiments include detection for example, of a wet diaper. Details are described below.

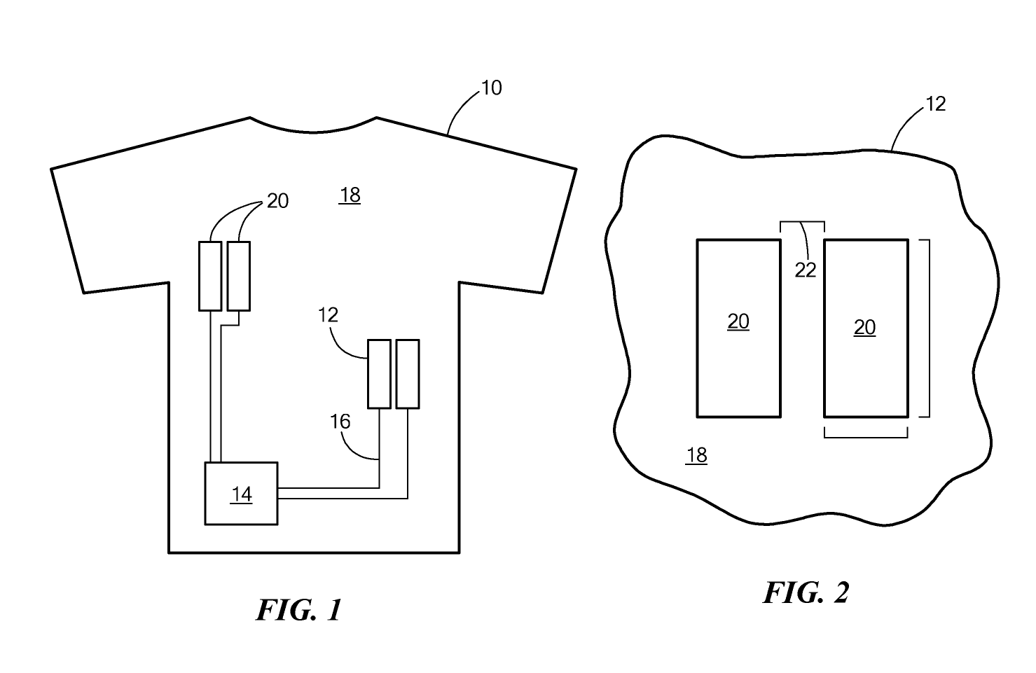

Referring now to the embodiments in more detail, in FIG. 1 there is shown an array of at least two conductive elements 20 that establish at least one sensor 12 mounted on an article of clothing 10, in accordance with an embodiment of the invention. Each sensor 12 may be further connected to a controller 14 through conductive traces 16. The sensor may be used, without limitation, to measure movement, although it is to be understood that other measurements are with the scope of the invention, such as a moisture condition.

The at least one sensor 12 that includes the two or more conductive elements 20 may be formed on a suitable elastic substrate 18, which may be implemented, without limitation, as an article of clothing 10, that can stretch and compress as a person breathes.

The article of clothing 10 may be sufficiently sized to fit an individual such that the substrate 18 will stretch elastically with a person's respiration but not be unnecessarily constricting. The conductive elements 20 may be so sized, without limitation, to allow for a change of capacitance on the order of five picofarads with a person's respiration. The conductive elements 20 may also be sized such that the total average magnitude of capacitance developed by the series capacitances of each conductive element, using the substrate 18 as a dielectric between the skin and each element, is on the order of fifteen picofarads.

The article of clothing 10 may be made of cotton, polyester, spandex, or any other sufficiently elastic material. In addition, article of clothing 10 may be shaped, without limitation, to the size of a shirt, a muscle shirt, and a shirt without sleeves, a suitable band around the chest, an infant's diaper, an infant's shirt or any other suitable shape to move with relation to the body. The article of clothing 10 may also be replaced by an elastic bandage such that the bandage constrained on a person's chest moves with respect to the body. Further, the various components on the article of clothing 10 may be made of different materials.

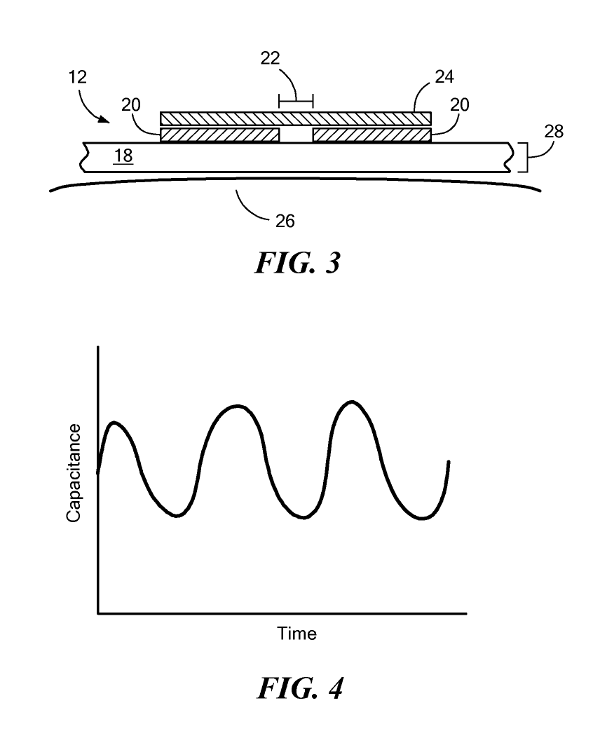

Referring now to the embodiments shown in FIG. 2 and FIG. 3, there is a movement sensor 12 composing of at least two conductive elements 20. The conductive elements 20 are positioned such that there is a suitable but fixed gap 22 between them. The conductive elements 20 may be attached, without limitation, to a non-elastic material 24 on one side and onto an elastic, dielectric substrate 18 on the other. The entire sensor is placed above a body part 26 at a suitable height 28

In more detail, still referring to the embodiments of FIG. 2 and FIG. 3, the conductive elements 20 may be optionally constrained to a gap 22 by a non-elastic, dielectric material 24. In this way, the capacitance formed between the elements 20 from the gap 22 is not able to change (depending, for example, on the dimensions of conductive elements 20, such constraint may not be desired and/or needed). The entire movement sensor 12 is placed over a body part 26 forming an electromagnetic field between the conductive elements 20 and the body part 26. As the distance 28 changes, the electrometric coupling between the body part 26 and the conductive elements 20 changes. This change in electromagnetic coupling can be read as a change in capacitance across the conductive elements 20.

In further detail, still referring to the embodiments of FIG. 2 and FIG. 3, the conductive elements 20 may be properly sized to have a total capacitance with the body part 26 near 15 picofarads, such that each conductive element has a height 21 of about 10 to 40 mm and a width 23 of about 10 to 40 mm. The distance 28 of the dielectric substrate may be, without limitation, of about 1 to 10 mm. In other embodiments, the distance 28 of the dielectric substrate may have, without limitation, a much larger length and/or capacitive output. For example, the dielectric substrate may cover a large portion of a subjects chest.

The dielectric substrate 18 shown in FIG. 2 and FIG. 3 may be made of any suitable material such as cotton, polyester, spandex, thin plastic, elastic vinyl, or any other such material. In addition, the conductive elements 20 may be made of any conductive material including but not limited to metal plates, metal films, conductive inks, conductive epoxies, conductive foams, and conductive plastics or vinyl. The non-elastic material 24 may be any material that restricts lateral movement of the conductive elements 20 including non-elastic fabrics, plastics and vinyl. In addition, the dielectric substrate 18 may include an adhesive bandage such that respiration will cause a compression of the bandage to bring the conductive elements 20 closer to the body part 26. Alternatively, the dielectric substrate 18 may include a loose fitting shirt such that respiration will cause the shirt to constrict against a person's chest, bringing the conductive elements 20 closer to the body part 26.

Referring back to FIG. 1, the controller 14 may be configured to provide an alarm based off of the measure of movement of the body part. For example, the body part may be the chest, and movement of the chest may be used to measure respiration, wherein the controller 14 provides an audio alarm based on a change in respiration. FIG. 4 is a graph showing an exemplary sample of respiration data gathered from an embodiment of the sensor shown in FIG. 2 and FIG. 3.

The controller 14 may be embodied in many different forms, including, but in no way limited to, computer program logic for use with a processor (e.g., a microprocessor, microcontroller, digital signal processor, or general purpose computer), programmable logic for use with a programmable logic device (e.g., a Field Programmable Gate Array (FPGA) or other PLD), discrete components, integrated circuitry (e.g., an Application Specific Integrated Circuit (ASIC)), or any other means including any combination thereof.

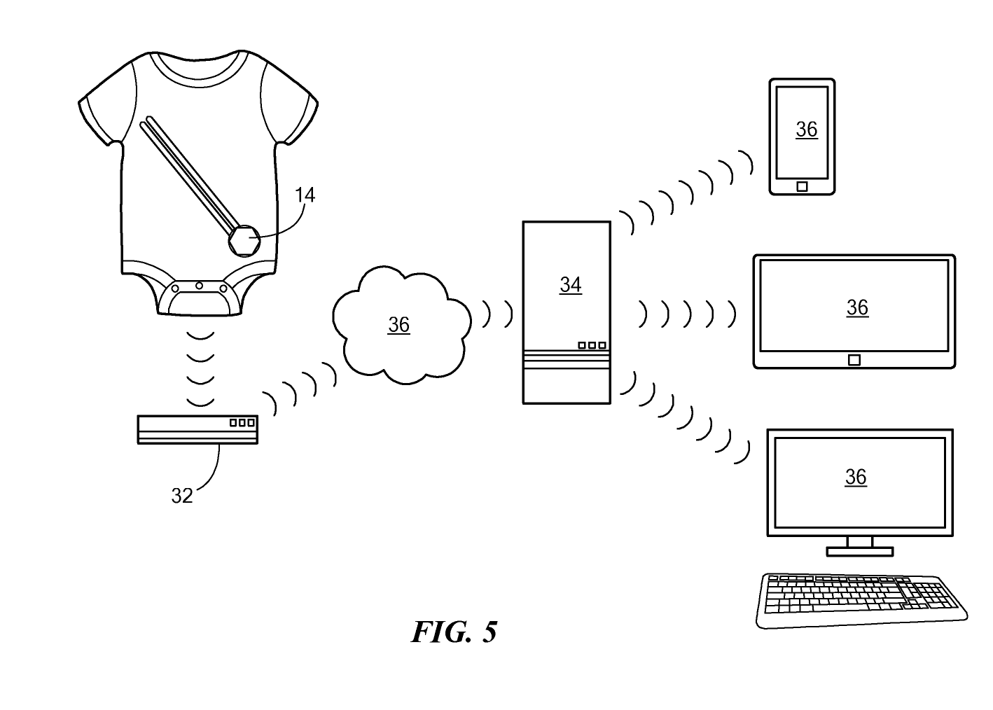

Referring now to the embodiment shown in FIG. 5, the controller 14 may be operatively coupled to a base station 32. For example, the controller 14 may be connected wirelessly to a base station 32 using, without limitation, Bluetooth technology. In other embodiments, the controller may be connected to the base station 32 using an appropriate cable. The base station 32 may be further operatively connected to the internet 38, either wirelessly or through appropriate cabling. In various embodiments, the base station 32 may be linked, without limitation, via the internet, to a server 34 that can receive and transmit data gathered by the controller 14. The data transmitted by the controller 14 to the server 34 may be substantially raw sensor data, with the controller 14 performing no, or substantially no, analysis on the data. In such embodiments, the server 34 may perform a majority or all of the data analysis. The server 34 may be configured to transfer data and/or alerts, based, at least in part, from the data received from the base station, to a personal computing device 36. For example, the personal computing device 36 may be connected to the internet, and may be configured to receive data and alerts from the server 34.

In various embodiments, the controller 14 may be configured to receive data from a sensor or an array of sensors. The sensor(s) may be, but is not limited to, the sensor depicted in FIG. 3. It is to be understood that any type of sensor may be utilized, that provides measurements or other parameters associated with the body part. For example, the one or more sensors may include, without limitation, an accelerometer (that provides, for example, position, orientation, and/or motion data), a MEMS sensor, and/or a temperature sensor. GPS information may be provided to the controller 14. Additionally, the controller may be configured to automatically connect wirelessly to the base station 32 such that data from an array of sensors is transferred to the base station 32. In this way, a low power transmitter can be used by the controller 14 to reduce energy and size requirements. The base station 32 may be further configured to automatically connect to a local wireless network. Once the base station 32 is connected to both the controller 14 and the internet 38, data collected by the controller 14 can be relayed to the server 34. In this way, a remote server can constantly receive data from an array of sensors operatively coupled to the controller 14. Additionally, the server 34 may be configured to analyze data transmitted from the controller 14 in real time, with the data relayed to a personal computing device in a human readable format. In addition, the server 34 may also be configured to analyze data transmitted from the controller 14 to detect abnormalities and signal alerts. Furthermore, the server 34 may be able to transfer information to a personal computing device 36 for displaying in real time. The server 34 may also be configured to save settings from the personal computing device 36 as well as transfer alerts detected from the controller 14 to the personal computing device 36. In this way, the server 34 can analyze data from the controller 14 substantially in real time and can send alerts in substantially real time to the personal computing device 36, even if the personal computing device is in a passive mode and not actively receiving data from the server 34.

In further embodiments, the controller 14 may be configured to gather data from an array of sensors over a period of time, such as, without limitation, 1 second, and subsequently provide periodic transmission to the base station 32.

The controller 14 may be attached to the array of sensors using a conductive and magnetic material such that a physical connection is maintained through the force of a magnetic field. Furthermore, the controller 14 may be operatively connected to the base station 32 through any suitable wireless connection such as a Bluetooth, a wi-fi connection, a FM/AM radio signal, through an infrared transmitter, through a zigbee or an network, or any other suitable wireless connection. Additionally, the base station 32 may include lights or speakers to alert a user. The base station 32 may also include a sound or video recording module and be configured to transmit this data to the server 34. The personal computing device may be, without limitation, a personal phone, a computer, a tablet, or any other suitable electronic device which is connected to the internet and can display an output.

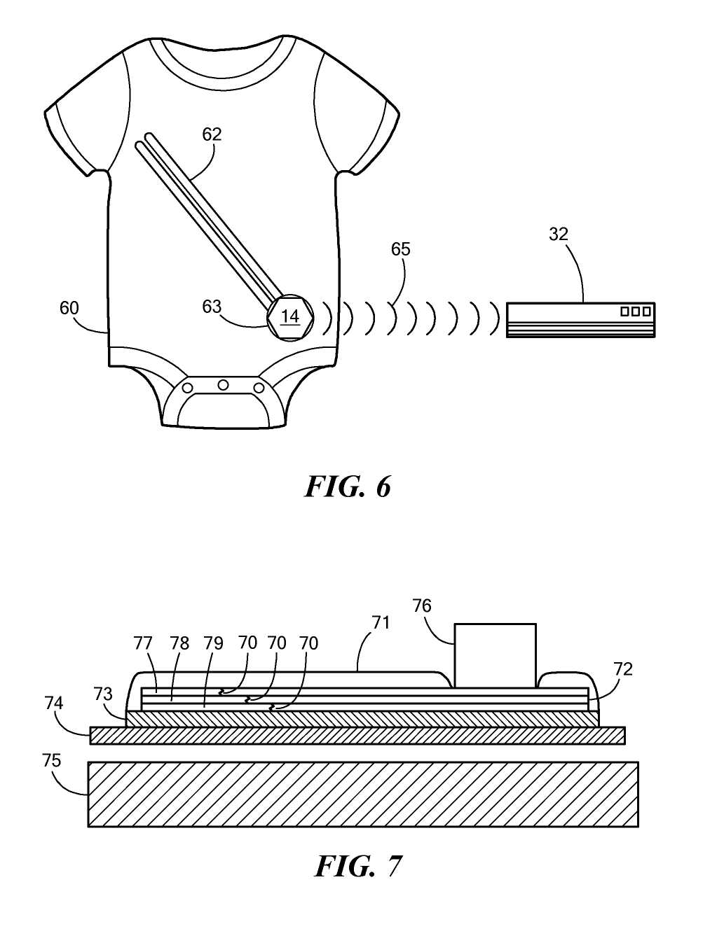

FIG. 6 shows a controller 14 coupled to an infant bodysuit 60, in accordance with an embodiment of the invention. For example, the bodysuit 60 may be, without limitation, a onesie. As shown in the embodiment of FIG. 6, the conductive elements 62 traverse over at least a portion of the chest area. To increase sensitivity, the conductive elements may have a length that spans across a large portion of the chest. For example, the conductive elements may have a length greater than a 25% the width of the chest (as measured from approximately 1″ below the armpit), a length greater than 50% the width of the chest, or a length greater than 75% the width of the chest. Using the above-described system, respiration sensing technology is combined with real-time data streaming and customizable alerts to ensure parents know their baby is breathing and healthy while they sleep. By using flexible, machine-washable, and non-contact sensors embedded onto a bodysuit, parents are provided with information regarding respiration, skin temperature, and body position, depending on the sensors implemented. Data may be sent wirelessly 65 to the base station 32 from the controller 14 (which, as shown, may be, without limitation, removably connected via a base station magnetic pad 63) that is attached to the bodysuit 60, where it can then be provided to, and analyzed at, a server, with notifications sent to a personal computing devices, such as a smartphone or tablet via the cloud, ensuring parents know both when their baby is well and when something is wrong.

FIG. 7 shows a cross-section of various components associated with a system for monitoring a body part, in accordance with an embodiment of the invention. A conductive element 72 is coupled to a body part 75 via a dielectric 74, which as described above may be an article of clothing. In other system embodiments, the dielectric may be, for example, an adhesive bandage or diaper, described in more detail below. Only a single conductive element is shown in FIG. 7, however it is to be understood that, for example, each respiration sensor may be composed primarily of two long conductive traces that are located near each other, as described above, about one inch apart.

The conductive element may be made of any conductive material known in the art. In various embodiments, a conductive ink may be used. The conductive ink may include a conductive metal, such as silver, nickel, copper, or gold, that is suspended in a solvent, such as, without limitation, ketone. For example, a conductive ink that may be used can include approximately 90% silver particles by weight suspended in a solvent that is primarily ketone. The conductive ink may be cured, for example, using heat or UV radiation.

In illustrative embodiments of the invention, each conductive element 72 may include multiple layers 77, 78 and 79 of conductive material bonded together so as to create an interface between the layers that impedes crack propagation from one layer to an adjacent layer. Advantageously, as the sensor is stretched and any one layer develops a crack 70, conductivity along the conductive element 72 is still maintained and is not broken. Thus, the sensor can be stretched by a much greater amount before failing.

To form the multi-layer conductive element 72, each layer of conductive material 77, 78 and 79 may be cured or flash cured/dried (i.e., partially cured) before depositing the next layer of conductive material. For example, during the flash cure, solvent is removed from a film layer 77, 78 and 79 causing the layer to harden. When each successive film layer is applied, the solvent in each new film layer helps to slightly integrate the new film layer into the film layer below but does not completely dissolve the lower film layer. In this manner, there is a blended interface between each film layer (similar to pouring a layer of concrete, letting the concrete almost dry, then pouring a new layer of concrete on top). For the most part, the film layers 77, 78, 79 are distinct and separate, however their interfaces are slightly mixed. Because of this, as the sensor is stretched, cracks and discontinuities that form in the layers have a hard time propagating through the interface and the sensor can be stretched to a much greater amount.

In an exemplary process not meant to be limiting, after each layer of conductive film is deposited, the layer is flash dried with approximately a 300 degree flash dryer for less than six seconds. This step removes the majority of the solvent from the layer and makes it fairly dry to touch. At this point, the layer is mostly a solid. Once all the layers are added the entire conductive element 72 goes into an oven at 125 degrees for approximately 20 minutes for a full curing process (manufacture specified). The full cure removes the rest of the solvent while maintaining the interface and the distinct flavor of the various layers.

Any number of layers may be deposited in forming the conductive element 72. In various tests, it was found that by flash curing each layer of conductive ink to produce three distinct bonded layers of conductive ink, the amount of strain before breaking conductivity increased from approximately 7% strain to greater than 20% strain. This increase in functional elasticity did not occur with either a single large layer of conductive ink or when multiple layers were applied but not partially cured before deposition of the next layer.

Still referring to FIG. 7, two exemplary fabrication methodologies are provided. In a first method, an insulator, such as a standard heat transfer t-shirt vinyl 71, is cut to the desired shape of the sensor. Depending on size, multiple sensors can be cut on the same sheet. The vinyl may be, without limitation, a thin, approximately 1 mm layer of vinyl. Such vinyl may include a plastic backing on one side, and/or a hot melt glue applied to the other depending on application.

Once cut, the vinyl 71 is placed on a silk screen machine where a thin layer of silver conductive ink is applied to the vinyl 71. The deposited ink may have slightly smaller dimensions than the outer perimeter of the vinyl trace associated with each sensor. While only one film layer is necessary, advantageously multiple layers of conductive ink may be applied sequentially, with each layer separately flash dried upon deposition to create distinct layers of conductive material 77, 78 and 79, as described above. After three layers are applied the entire sheet is placed in an oven to fully cure the silver ink. A mounting element/pad 76 for a controller, which may be, without limitation, magnetic is integrated with the conductive element so as to be operatively coupled with the conductive element 72.

At any point in the process a separate but matching layer of vinyl 73 is heat pressed to the desired dielectric 74. Once the sensors are cured, the vinyl layer 71 that includes the conductive element 71 is heat pressed on top of the vinyl layer 73 such that the conductive element is completely encased and insulated by the vinyl.

In a second exemplary process, a vinyl layer is cut to shape and one or more layers of conductive ink is applied, as described above. A final layer of thick plastic based ink is screened on top of the conductive layers. Once fully cured, a hot melt glue is applied to the top ink layer and the entire sensor is heat applied directly to the desired dielectric.

Upon receiving at least one time-varying electrical input signal characterizing respiratory activity of a subject, such as from, without limitation, the movement sensor of the above-described embodiments, an abnormal respiratory event may be detected. Analysis of respiratory activity and detection of the abnormal respiratory event may occur at any stage/component of FIG. 5, including at the controller 14, the base station 32, the server 34, and/or the personal computing device 36. Upon detection of an abnormal respiratory event, such as apnea or sudden infant death syndrome (SIDS), an alert may be generated at a user interface (such as, without limitation, the personal computer device 36).

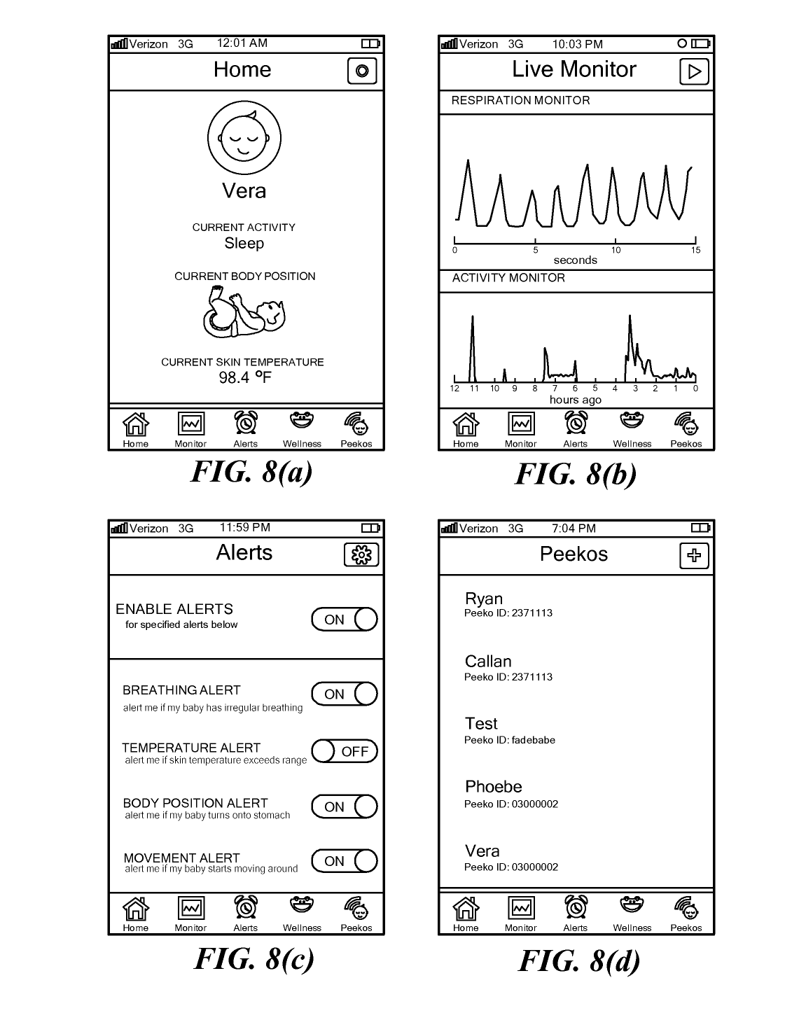

FIGS. 8(a-d) show exemplary screen shots that may be displayed on a personal computer device based, at least in part, on the received sensor(s) information, in accordance with various embodiments of the invention. More particularly, FIG. 8(a) shows a home display mode, in which relatively general information pertaining to a subject is displayed. Such information may include, for example, current activity (e.g., awake or sleep), body position (e.g., laying on chest or back), and skin temperature. Other display modes, such as, without limitation, monitor, alert setting, wellness, and peekos, may be viewed by selecting the appropriate button at the bottom of the screen. FIG. 8(b) shows a live monitor mode, in which contemporaneous data from the sensors may be displayed and/or various alerts. For example, and without limitation, current respiration data and activity data may be viewed. FIG. 8(c) shows an alert setting mode, which allows the user the capability to select and enable various alerts, such as breathing, temperature, body position and movement alerts. FIG. 8(d) shows a controller set up mode, which allows the user the capability to select and configure one or more controllers.

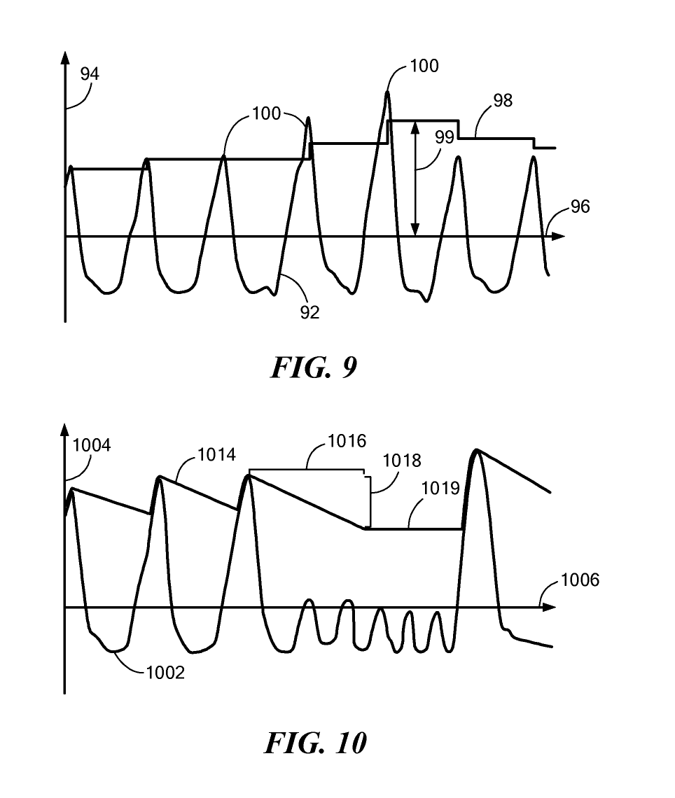

FIG. 9 shows an input signal 92 characterizing respiratory activity changing in amplitude 94 over time 96, in accordance with an embodiment of the invention. The peaks 100 of the input signal 92 are also shown. In addition, a time-varying segmented following signal is shown 98, each segment corresponding to a segment of the input single 92. In the embodiment of FIG. 9, the following signal 98, without limitation, changes over time and, at least in part, follows the envelope of the input signal 92.

As shown in FIG. 9, the following signal 98 may be calculated such that the value of the following signal 98 changes over time 96 based on the running average of the peaks 100. In this way, the following signal 98 follows the general shape of the peaks 100 while minimizing the effects of large or rapid changes.

Upon determining a peak 100 in the input signal 92, a segment of the following signal 98 may be generated, without limitation, such that the magnitude of its value 99 stays constant over time 96 when the input signal 92 is not a peak 100. Upon determination of the next peak in the input signal 92, a new segment of the following signal 98 may be generated, with the magnitude 99 of the following signal 98 calculated such that the new magnitude 99 is again based off of the running average of peaks 100. In various embodiments, the magnitude 99 of the following signal 98 may be calculated such that the magnitude 99 stays constant while the signal 92 is not at a peak and such that the change in magnitude 99 is less than the absolute change between peaks 100.

FIG. 10 shows an input signal 1002 characteristic of respiration changing in amplitude 1004 over time 1006 that includes an apnea event, in accordance with an embodiment of the invention. The following signal 1014 includes segments that, similar to FIG. 9, are triggered by a peak in the input signal 1002. Each segment includes a calculated slope such that the segment decreases by magnitude 1018 for a determined time 1016.

As shown in FIG. 10, each segment of the following signal 1014 may be calculated such that while the input signal 1002 is above the following signal 1014, the magnitude of the segment is equal to the input signal 1002. In addition, the magnitude of each segment may be calculated such that while the input signal 1002 is below the following signal 1014, the following signal 1014 decreases by a certain amount 1018 over a certain time 1016. In various embodiments, after a certain period of time, the magnitude of a segment may stop decreasing and, for example, level off 1019.

If the input signal 1002 is still below a specific segment of the following signal 1014 after a predetermined time threshold 1016, an apnea event and/or SIDS event may be detected. An abnormal respiratory event may then be stored that is associated with a segment in the input signal corresponding to the specific segment. Various audio, visual, and/or vibration alerts may consequently be generated at, for example, the personal computing device. Alternatively, if the input signal 1002 rises above the corresponding segment of the following signal 1014 within the threshold duration, no abnormal respiratory event may be associated with the segment. A new segment of the following signal may start upon determination of the next peak in the input signal 1002.

In further detail, still referring to FIG. 10, when the input signal 1002 is determined to be below the following signal 1014, an exemplary calculation to determine a segment of the following signal 1014 is as follows:

New Value=(Last Value)−(drop 18)÷(time 16×sample rate).

In generating the linearly decreasing segment, the rate of change may be calculated, for example, by the last peak 1000, an average based off the input signal 1002, an average of the peaks 1000, or any predetermined or empirical value. The predetermined time 1016 and drop in magnitude 1018 may be computed, without limitation, based off of empirical studies or from values based on the signal 1002. The linearly decreasing segment may also be calculated such that a decrease is triggered from the input signal 1002 or a secondary function, such as the envelope of the input signal 1002.

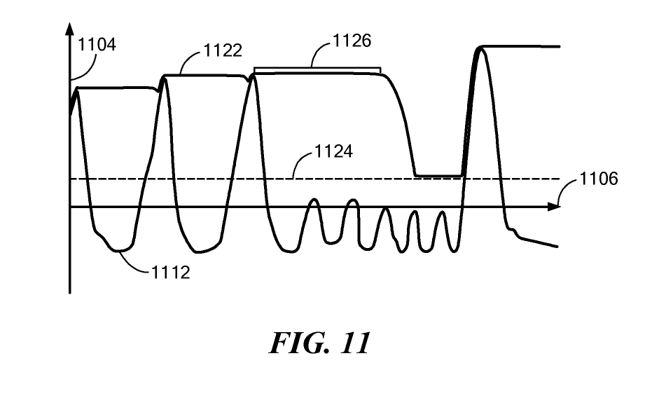

Referring now to FIG. 11, there is shown an input signal 1112 characteristic of respiration including an apnea event changing in amplitude 1104 over time 1106, in accordance with an embodiment of the invention. An example of an exponentially decreasing segment of the following signal 1122 is shown that decreases only after a time 1126 from a detected peak of the input signal 1112. In addition, a predetermined magnitude threshold 1124 is shown. The exponentially decreasing segment may be calculated such that while the input signal 1112 is above the magnitude of the following signal 1122 segment, the magnitude of the segment is equal to the input signal 1112. If a segment of the following signal 1122 decreases past a threshold 1124, an apnea and/or SIDS event may be counted. Alternatively, as in the above-described embodiment, an apnea and/or SIDS event may be detected if the input signal 1112 is still below a specific segment of the following signal 1122 after a predetermined time threshold.

When the input signal 1112 is determined to be below a segment of the following signal 1122, a general example for calculating a new value of the segment of the following signal 22 may be as follows:

New Value=starting magnitud [symbol] ee^{time×drop rate}

In the above equation, the time may be the number of samples since the input signal 1112 has dropped below a segment of following function 1122. The starting magnitude may be computed from the magnitude of the input signal 1112 when it first drops below the segment of the following signal 1122. The starting magnitude may also be based off a predetermined value, a separate function, or any other value. The drop rate may be computed as:

drop rate=In(starting magnitude÷(sample rate×time)).

In various embodiments, the drop rate may be based off of a predetermined time or based off a value found through the average of the input signal 1112.

Because a second signal (i.e., the following signal) is defined that responds to, but is not identical to the original respiration signal, variations are allowed to occur in the respiration signal that still may track to an abnormal respiration event. For example, in Cheyne-Stokes respiration, the respiration amplitude slowly decreases over time. With a standard amplitude/time threshold, an event may not be counted because the majority of the input signal might not be below a magnitude threshold. Because the following signal is based off of the last major peak, and allows for anticipated variations, situations such as Cheyne-Stokes respiration would show up as an accurate event.

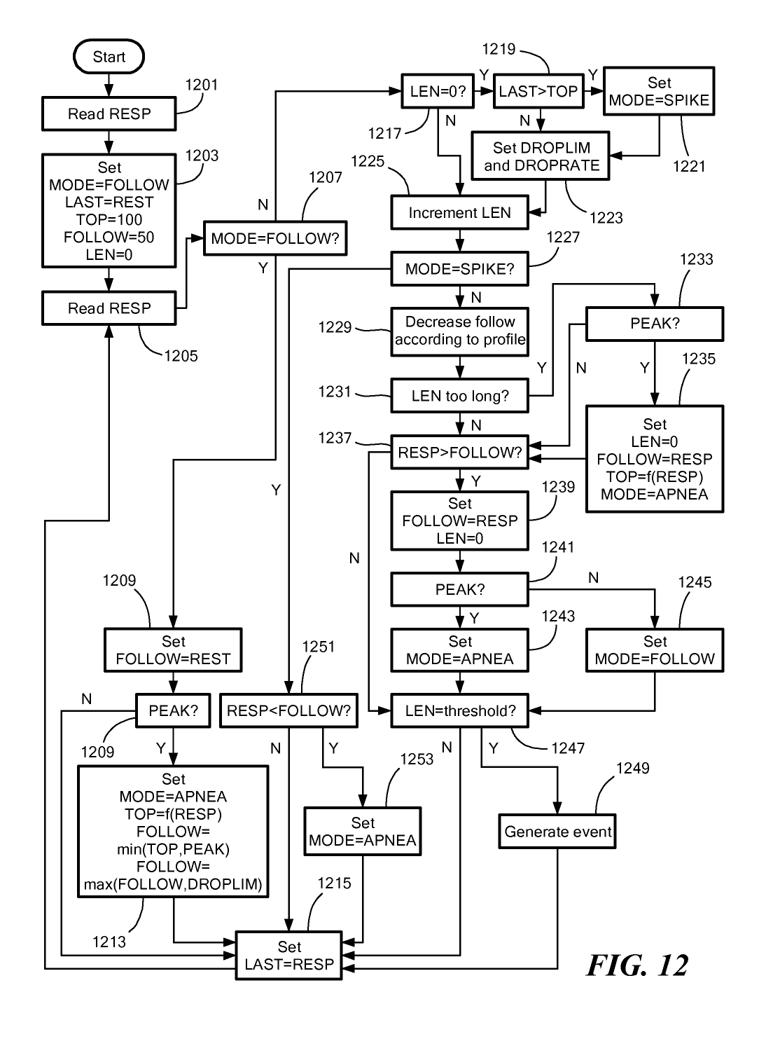

FIG. 12 shows a flow diagram depicting an exemplary process of detecting an abnormal respiration event, in accordance with an embodiment of the invention. As shown, the methodology may, without limitation, incorporate a state machine that allows detection of apnea with each new incoming sample.

When the process is first started, the first respiratory sample is read, step 1201, with the state (Mode) set to a “following” state and the variables (Top, Last, Follow, Len) set to initial values, step 1203. The variable Follow is the magnitude of the following signal and is used to decide if an event has occurred, as described above.

Once a new sample is read in, step 1205, if Mode equals following, step 1207, the Follow variable is set to the current respiration value, step 1209, and a check is performed to see if the current sample is a peak, step 1211, using a peak detection algorithm as known in the art. If the current sample is a peak then the Mode switches to “apnea” and the Follow variable is changed to, without limitation, the minimum of the current respiration value or an average of all past peaks (but will not be set below a set value—“Drop Limit”), step 1213, and the Last variable is set to the current respiration, step 1215.

Once the mode is switched to “apnea”, a new sample is read, step 1205, and the process moves over to the other branch in the state machine. If the duration in the apnea branch is 0 (i.e., the first sample in the new mode/segment), step 1217, a check is performed to see if the new sample is less than the variable “top” (which equals, for example, the running average of past peaks), step 1219. If the new sample is more than the running average of past peaks the mode is set to “Spike”, step 1221. The Spike mode is used to make sure that the algorithm isn't running on bad data caused by movement spikes, and helps make the algorithm more robust to real world data. The variables “Droplim” and Droprate”, used in determining the following signal may then be set, step 1223.

After this check for the Spike mode, the Len variable (i.e., a count of the number of samples associated with the duration of the current segment of the following signal) is incremented, step 1225. If the state is in “spike” mode, step 1227, the new Resp value is compared with the Follow variable to see if the data is no longer a spike, step 1251. If no longer a spike then the mode is switched back into “Apnea” mode, step 1253. If, at step 1227, the state is in “Apnea” mode, then the next Follow value is created by acting on the last Follow value according to a desired profile, step 1229. As discussed above, this profile may be, without limitation, a linear decrease, an exponential decrease or any other profile (typically decreasing and/or remaining the same over time). Next the Len variable is compared to see if it is above a predetermined value, step 1231. If the Len is larger than the predetermined value, step 1231, then it may be an indication that an event already detected (e.g., an apnea event) is unnaturally long and likely not an apnea event. This could be the result of bad data or that the subject has stopped breathing (e.g., SIDS, in which case a SIDS event could be set). If the Len is too long then the state may wait until the next marked peak, step 1233, and resets all of the values and state back to the apnea state, step 1235.

If the Len hasn't gone over that max value then the algorithm checks to see if the current Resp value is above the Follow value, step 1237. If so, then there is no event and the algorithm goes through a check and resets the variables, step 1239, and sets the state Mode back to either “follow”, step 1245, or “apnea”, step 1243, depending on if the respiration data is currently a peak, step 1241.

If the Resp value is below the Follow value, step 1237, the process checks to see if the Len is past the duration threshold set for an Apnea event, step 1247. If so, an event is generated, step 1249. If an event is generated the Mode stays the same—this allows the process to continue to see if the event goes on too long (see step 1231) at which point the event is disqualified (or the determination of, for example, SIDS).

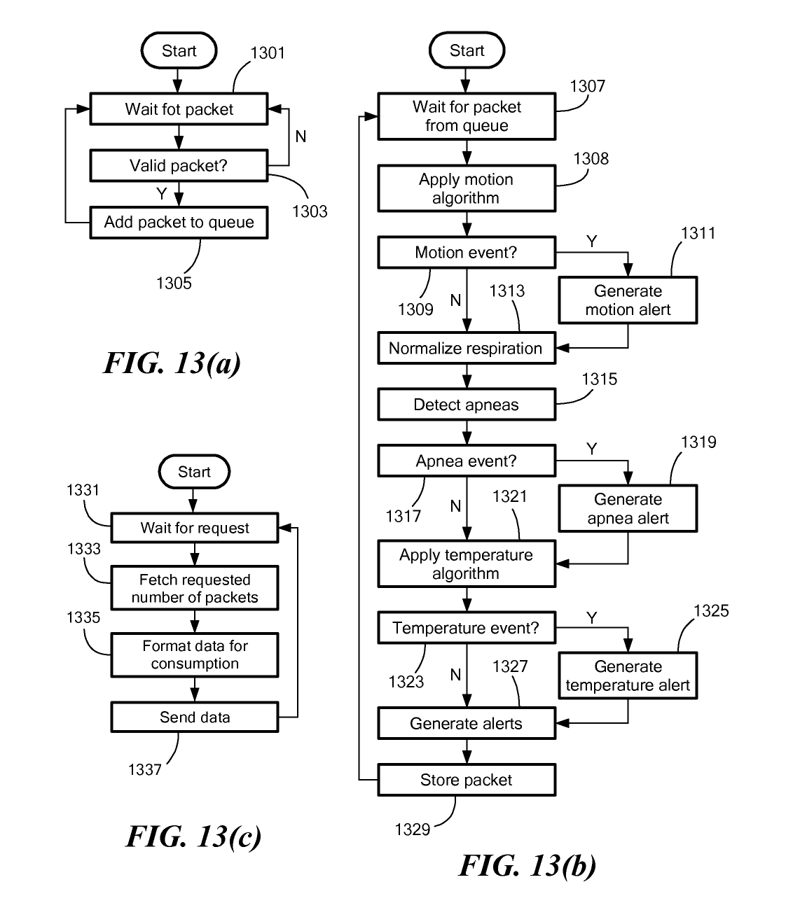

FIGS. 13(a-c) show exemplary flow diagrams depicting how data is transferred into and out of the server (see, for example, FIG. 5), in accordance with various embodiments of the invention. More particularly, FIG. 13(a) is a flow diagram depicting how data flows from the devices (i.e., sensors, controllers, and/or base stations) into the server. FIG. 13(b) is a flow diagram showing how the server handles running algorithms, storing data, and storing algorithm outputs. FIG. 13(c) is a flow diagram showing how the server handles communication with personal computing devices and sending alerts to users. The process presented in FIGS. 13(a-c) advantageously allows computing power to be scaled independently of how many devices or personal computing devices are trying to connect to the server. For example, if many more devices compared to personal computing devices suddenly attempt to connect to the server, the processes shown in FIGS. 13(a-b) can be scaled without increasing the processes associated with FIG. 13(c).

Referring to FIG. 13(a), the server waits for an incoming packet from a sensing device which typically includes an identifier of what device it came from as well as whatever data is being transmitted, step 1301. The server then checks to make sure the packet is valid and contains valid data, step 1303. If the packet is valid then the data, including identifier, is added to a memory queue that can be accessed by the other sections, step 1305.

Referring to FIG. 13(b), the server first checks to see if there is new data in the queue, step 1307. If there is data in the queue then a motion algorithm is applied, step 1308, to check if there is motion (e.g., motion of the body part due to respiration), step 1309. If there is motion then a motion alert is generated, step 1311. Next the respiration data is normalized, step 1313 and an apnea algorithm is applied, step 1315. If apnea (or SIDS) is detected, step 1317, then an alert is generated, step 1319. Next the temperature (or any other data from any additional sensors) is checked, step 1321, and if an abnormal temperature is detected, step 1323, a temperature alert is generated, step 1325. If any alerts were generated, the alerts are passed into a memory queue that is checked and generates alerts to the users (e.g., personal computing devices) depending on the alert method, step 1327. Lastly, the raw data as well as the algorithm results may be stored in, without limitation, server memory, step 13297.

Referring now to FIG. 13(c), the server waits for a request from a personal computing device, step 1331. The system then fetches the requested data from long term memory, step 1333, and then formats the data for the personal computing device, step 1335. Lastly, the system transmits the formatted data to the personal computing device and waits for further requests, step 1337.

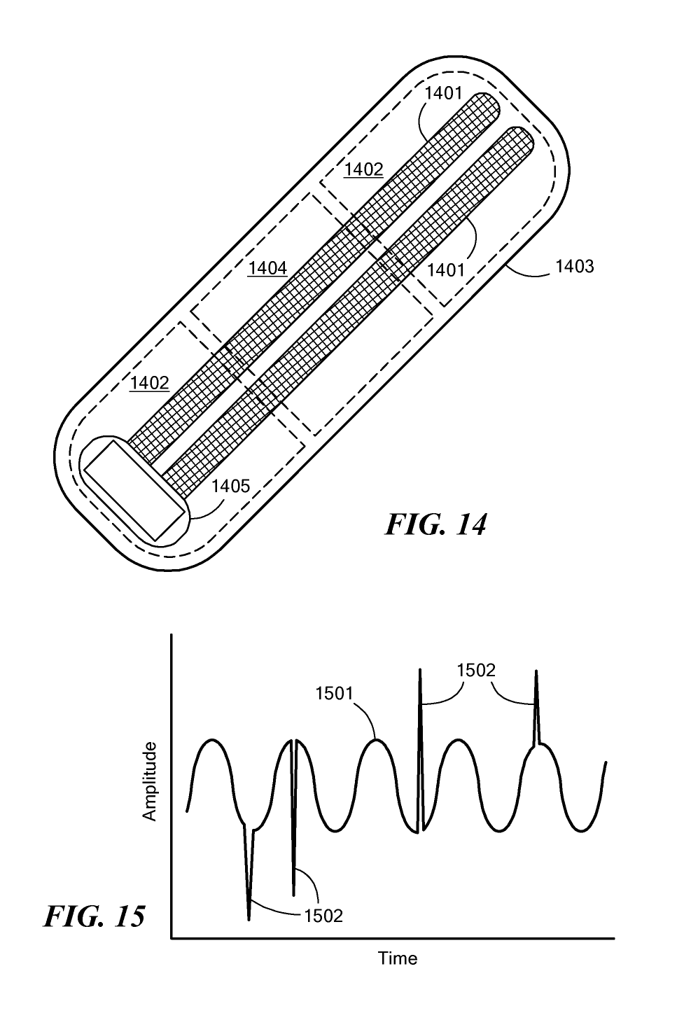

Because of above-described sensors ability to pick up extremely small capacitive changes, it may be unnecessary for the article of clothing (shown, for example, in FIGS. 1 and 6) to go all the way around the body part being measured. Instead, small changes may be found from smaller conductive elements on a bandage that can be attached directly to a body part. FIG. 14 shows an adhesive bandage that may include one or more sensors, in accordance with an embodiment of the invention. The adhesive bandage may include, without limitation, a base layer 1403 that may include a rectangular or other shaped patch of material, such as a plastic, a vinyl, and/or a tightly woven fabric. On top of this base layer 1403 the above-described methodologies of applying the sensor materials may be followed to produce two conductive elements 1401 that may extend, without limitation, along the longest dimension of the base layer 1403. The conductive elements 1401 may be further coupled to a controller 1405. Under the base layer 1403, a temporary adhesive 1402 may be attached to both ends of the base layer 1403, such that each end of the base layer 1403 will stick to the body. Furthermore, under the base layer 1403, the middle portion between the temporary adhesives may be covered in a compressible, elastic material 1404 such as foam or gauze. The elastic material 1404 may illustratively be 3-10 mm thick.

While measuring respiration, it is often advantageous to attach the bandage to the area of greatest movement such as on the ribs or stomach. During respiration, the chest increases and decreases in diameter. Because the two ends of the base layer 1403 are attached to the skin, the base layer 1403 is stretched. As the base layer 1403 is stretched, the compressible material 1404 under the base layer 1403 is compressed. This compression brings the conductive elements 1401closer to the body and increases the capacitance output to the controller 1405. The bandage may be particularly useful whenever it is not possible to fully encompass a body part. Specifically for respiration, this may include situations in a hospital when a patient has multiple electrical leads attached to their body and a band or shirt can not conveniently be worn. In addition, the bandage may be useful in radiation-oncology lab or any other situation(s) in which a patient is going through image scanning, such as a CT scan or PET scan, while respiration data is required.

The above-described sensor may also be used in a wide variety of application, including, without limitation, a system for detecting wetness associated with an article of clothing, such as diaper, in accordance with an embodiment of the invention. To detect a wet diaper, the above-described sensor may be configured such that it wraps around the diaper (groin) region. In addition to being extremely sensitive to the capacitive system that includes each conductive element and the body, the sensor is also very sensitive to any changes in the insulating dialectical material between each trace and the body. Because water, feces, and urine have a significantly different electrical conductivity than air, as a diaper is being soiled, the composition of the dialectic in the capacitive system changes. As a diaper is being soiled, it can be measured as an increase in the total capacitance between the two conductive elements. Because this increase in capacitance may be masked by movement, the sensor location and/or configuration should be taken into account so as to advantageously minimize the effects due to body movement, respiration, and/or to accurately measure base levels to notice any change.

Over time, the above-described sensors may degrade, primarily due to excess strain during use as well as during normal wash cycles. While the vinyl insulation layers (see, for example, FIG. 7 and accompanying text) may deteriorate over time, mechanical separation of the internal conductive layer(s) associated with the conductive elements is of greatest concern.

Because the conductive material is less elastic than the insulating vinyl layers, when excess strain is placed on the sensor, micro fractures occur across the conductive material. Typically, when the strain is removed, the two parts of the micro fracture maintain contact and the material remains conductive. However, when strain is again placed on the sensor, the two sides of the micro fracture separate and no longer conduct an electrical signal. As described above (see, for example, FIG. 7 and associated text) reliability may be increased if multiple layers of conductive material are used for the conductive element, that are bonded together so as to create an interface between the at least two layers that impedes crack propagation from one layer to an adjacent layer. The performance of the sensors is highly dependent on the area of the conductive trace. When a micro fracture separates a trace, the total area of active and conductive material is dramatically reduced. Depending on the level of strain on the sensor while it is being worn, the micro-fractures can alternate between conductive and non-conductive states as a person breathes and adds or removes strain from the sensor. This alternation of conductive states shows up as an extremely large level shift 1502 in the resulting capacitance of the sensor at either the peak or valley of the respiration signal 1501, as shown in FIG. 15, in accordance with an embodiment of the invention. In a very mild case, a short but high amplitude peak is observed occasionally. In more problematic cases, a short but high amplitude peak is observed during most breathing periods. In a very bad case, the signal is heavily distorted at the same period of breathing.

Sensor degradation can be found by searching for short but periodic spikes in the sensor output data. The amplitude of the spikes is almost always the full range of the capacitance sensing method. While similar spikes can occur from normal movement, spikes caused from sensor degradation are highly regular and have a period very similar or identical to the respiration period.

Computer program logic implementing all or part of the functionality previously described herein may be embodied in various forms, including, but in no way limited to, a source code form, a computer executable form, and various intermediate forms (e.g., forms generated by an assembler, compiler, linker, or locator.) Source code may include a series of computer program instructions implemented in any of various programming languages (e.g., an object code, an assembly language, or a high-level language such as Fortran, C, C++, JAVA, or HTML) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies, networking technologies, and internetworking technologies. The computer program may be distributed in any form as a removable storage medium with accompanying printed or electronic documentation (e.g., shrink wrapped software or a magnetic tape), preloaded with a computer system (e.g., on system ROM or fixed disk), or distributed from a server or electronic bulletin board over the communication system (e.g., the Internet or World Wide Web.)

Hardware logic (including programmable logic for use with a programmable logic device) implementing all or part of the functionality previously described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL.)

The embodiments of the invention described above are intended to be merely exemplary; numerous variations and modifications will be apparent to those skilled in the art. All such variations and modifications are intended to be within the scope of the present invention.

What is claimed is:

1. A method of monitoring a body part of a subject, the method comprising:
mounting on the body part, via at least one dielectric, at least two conductive elements in a manner wherein the distance between the conductive elements and the body part varies with movement of the body part, each conductive element including at least two layers of conductive material bonded together so as to create an interface between the at least two layers that impedes crack propagation from one layer to an adjacent layer; and
measuring changes in capacitance between the elements and the body part that result from movement of the body part.

2. The method according to claim 1, wherein the at least two layers of conductive material include a first and second film layer, the second film layer deposited onto the first film layer after curing and/or flash curing the first film layer, whereupon the second film layer is cured and/or flash cured so as to form the bond therebetween.

3. The method according to claim 1, wherein one or more of the at least two layers of conductive material include conductive ink.

4. The method according to claim 1, wherein measuring changes in capacitance includes measuring changes in a series capacitance along a path that includes each conductive element and the body part.

5. The method according to claim 1, wherein the dielectric is an elastic substrate that is mounted on the body part.

6. The method according to claim 1, wherein the body part is the chest, and movement of the chest is used to measure respiration.

7. A system for monitoring a body part of a subject, the system comprising:
at least one dielectric that is mountable on the body part;
at least two conductive elements coupled to the at least one dielectric, the at least two conductive elements including an a capacitive output reflective of capacitance between the elements and the body part, each conductive element including at least two layers of conductive material bonded together so as to create an interface between the at least two layers that impedes crack propagation from one layer to an adjacent layer; and
wherein the dielectric is configured so that distance between the conductive elements and the body part varies with movement of the body part so as to produce corresponding changes in the capacitive output.

8. The system according to claim 7, wherein the at least two layers of conductive material include a first and second film layer, the second film layer deposited onto the first film layer after curing and/or flash curing the first film layer, whereupon the second film layer is cured and/or flash cured so as to form the bond therebetween.

9. The system according to claim 7, wherein one or more of the at least two layers of conductive material include conductive ink.

10. The system according to claim 7, wherein the dielectric is an elastic substrate that is mounted on the body part.

11. The system according to claim 10, wherein the elastic substrate is an article of clothing worn by the subject.

12. A system for monitoring a body part of a subject, the system comprising:
an article of infant clothing;
at least one sensor coupled to the article of clothing, the sensor providing output resulting from movement of the body part, the movement associated with respiration;
a controller coupled to the substrate and operatively coupled to the at least one sensor, the controller for transmitting data associated with output from the sensor;
a base station for transmitting data received from the controller associated with output from the sensor;
a network server, the network server for receiving and analyzing data received from the base station associated with output from the sensor, determining a condition chosen from the group consisting of apnea and SIDS, and transmitting alerts pertaining to the condition; and
a personal computing device having a user interface, the personal device for receiving alerts from the network server pertaining to the condition, and providing the alerts to a user via the user interface.

13. A method of fabricating a sensor, the method comprising:
coupling to a dielectric at least two conductive elements forming a capacitive output, each conductive element formed by:
depositing a first layer of conductive material, the first layer including solvent;
at least partially curing the first layer such that at least a portion of the solvent in the first layer is removed;
depositing on the at least partially cured first layer a second layer of conductive material, the second layer including solvent; and
at least partially curing the second layer such that at least a portion of the solvent in the second layer is removed, wherein an interface is formed between the first and second layer that impedes crack propagation from the first layer to the second layer.

14. The method according to claim 13, wherein the conductive material is conductive ink.

15. The method according to claim 13, wherein the dielectric is elastic such that respiration of the infant causes the dielectric to stretch.

16. The method according to claim 13, wherein the dielectric is a diaper.

17. The method according to claim 13, wherein the dielectric is an adhesive bandage.

18. A method for determining the occurrence of an abnormal respiratory event in a subject, the method comprising:
receiving at least one time-varying electrical input signal characterizing respiratory activity;
generating a time-varying segmented electrical following signal wherein each segment thereof corresponds to a segment of the input signal;
electronically monitoring the following signal to identify a specific segment thereof having a duration in excess of a threshold, and if the specific segment has been identified, then storing an abnormal respiratory event associated with a segment in the input signal corresponding to the specific segment.

19. The method according to claim 18, wherein generating the following signal includes:
determining a peak in the input signal; and
upon determining a peak in the input signal, generating a segment of the following signal.

20. The method according to claim 19, further comprising:
determining if the input signal rises above the corresponding following signal within the threshold duration, wherein no abnormal respiratory event is associated with the segment.

21. The method according to claim 20, wherein upon determining that the input signal rises above the corresponding following signal within the threshold duration, the method further includes:
ending the segment;
determining a peak in the input signal; and
upon determining a peak in the input signal, generating another segment of the following signal.
